(19) United States
(12) Patent Application Publication
(10) Pub. No.: US 2024/0252165 A1
(43) Pub. Date: Aug. 1, 2024

(54) Medical Suture And Medical Suture Device

(71) Applicant: Genesis Medtech Surgical Device (Wuxi) Co., Ltd, Wuxi (CN)

(72) Inventors: Naijun Ge, Jiangsu Wuxi (CN); Luda Cheng, Jiangsu Wuxi (CN)

(73) Assignee: Genesis Medtech Surgical Device (Wuxi) Co., Ltd, Wuxi (CN)

(21) Appl. No.: 18/560,472

(22) PCT Filed: Feb. 15, 2023
(86) PCT No.: PCT/CN2023/076127

§ 371 Date: Nov. 13, 2023

(30) Foreign Application Priority Data
Feb. 25, 2022 (CN) 202210177012.6
Feb. 25, 2022 (CN) 202220404808.6

(51) Int. Cl. A61B 17/06

(52) U.S. Cl. CPC A61B 17/06166; A61B 2017/06057; A61B 2017/06176

(57) ABSTRACT

The present invention discloses a medical suture and a medical suture device, and belongs to the field of medical instruments. The medical suture comprises: a suture body, the suture body defining a longitudinal center line; and a plurality of pairs of barbs arranged at intervals along a direction of the longitudinal center line, each pair of barbs being symmetrically arranged on opposite sides of the suture body, the barb being provided with a first end connected with the suture body and a second end far away from the suture body, and a ratio of a transverse distance L1 of second ends of each pair of the barbs along a direction perpendicular to the longitudinal center line to a transverse dimension L0 of the suture body perpendicular to the direction of the longitudinal center line ranging from 1.5 to 2.5.

The present application claims priority of two CN applications 202210177012.6 and 202220404808.6, both filed on Feb. 25, 2022 with the CNIPA and titled “Medical suture and medical suture device”, and the whole contents of the two CN applications are incorporated herein by reference.

### TECHNICAL FIELD

The present invention relates to the technical field of medical instruments, and more particularly, to a medical suture and a medical suture device.

### BACKGROUND

A surgical suture is used for seaming wounds and operative incisions, and used for repairing damaged or cut muscles, blood vessels and tissues. An existing surgical suture comprises a suture with barbs, which is designed to be provided with protruding barbs on a suture body. The barbs are designed to be convenient to penetrate through tissues when pulled in a first direction, and to keep the tension of tissues when pulled in an opposite second direction. These barbs enable the suture to be used for seaming wounds, closing tissues, tensioning tissues and attaching a repair device without knotting. The suture with the barbs may be properly tensioned and fixed by applying tension to the suture.

In an existing suture with barbs, the barbs generally have the problems of insufficient structural strength, insufficient tissue fitting, and large tissue trauma caused by large barbs. On this basis, the present application is put forward.

### SUMMARY

Therefore, a first technical problem to be solved by the present invention is that an existing medical suture causes large tissue trauma.

A second technical problem to be solved by the present invention is that the existing medical suture has a low structural strength.

A third technical problem to be solved by the present invention is that the existing medical suture and tissues are a poor fit.

Aiming at the above technical problems, the present invention provides the following technical solutions:

A medical suture comprises: a suture body, wherein the suture body defines a longitudinal center line; and a plurality of pairs of barbs arranged at intervals along a direction of the longitudinal center line, wherein each pair of barbs is symmetrically arranged on opposite sides of the suture body, the barb is provided with a first end connected with the suture body and a second end far away from the suture body, and a ratio of a transverse distance L1 of second ends of each pair of the barbs along a direction perpendicular to the longitudinal center line to a transverse dimension L0 of the suture body perpendicular to the direction of the longitudinal center line ranges from 1.5 to 2.5.

In some embodiments of the present invention, a cross section of the suture body in an area without the barbs is approximately rectangular, and the suture body comprises a first end face and a second end face which are oppositely arranged and a third end face and a fourth end face which are oppositely arranged, wherein the barbs are connected onto the first end face and the second end face, a width D1 of the first end face ranges from 0.2 mm to 0.6 mm, and a width D2 of the third end face ranges from 0.2 mm to 0.6 mm.

In some embodiments of the present invention, the cross section of the suture body in the area without the barbs is approximately square.

In some embodiments of the present invention, a cross section of the suture body in an area with the barbs is approximately spindle-shaped.

In some embodiments of the present invention, the barb comprises an outside face and an inside face, the outside face extends to the second end of the barb along a first part of the suture body, the inside face extends to the second end of the barb along a second part of the suture body, and a ratio of a longitudinal dimension L2 of the barb along the direction of the longitudinal center line to the transverse dimension L0 of the suture body perpendicular to the direction of the longitudinal center line ranges from 1.8 to 2.8. In some embodiments of the present invention, a distance L3 between the first part and the second part along the direction of the longitudinal center line ranges from 0.4 mm to 1 mm.

In some embodiments of the present invention, the outside face is approximately an arc face, and the inside face is approximately a straight face.

In some embodiments of the present invention, a radius r1 of the arc face of the outside face ranges from 1 mm to 2 mm.

In some embodiments of the present invention, an included angle between an extension face of the inside face and the longitudinal center line ranges from 25 degrees to 45 degrees.

In some embodiments of the present invention, the outside face and the inside face are in transition through a first arc face, and a radius r2 of the first arc face ranges from 0.02 mm to 0.05 mm.

In some embodiments of the present invention, the outside face and the suture body are in transition through a second arc face, and a radius r3 of the second arc face ranges from 0.5 mm to 1.5 mm.

In some embodiments of the present invention, the inside face and the suture body are in transition through a third arc face, and a radius r4 of the third arc face ranges from 0.02 mm to 0.05 mm.

In some embodiments of the present invention, the barbs are equidistantly distributed along an extension direction of the longitudinal center line, and a distance T between adjacent barbs ranges from 1 mm to 2.5 mm.

In some embodiments of the present invention, the barbs are unequidistantly distributed along an extension direction of the longitudinal center line, and a distance T between adjacent barbs ranges from 1 mm to 2.5 mm.

In some embodiments of the present invention, the barbs extend towards the same direction to form a unidirectional barb suture.

In some embodiments of the present invention, the suture body forms a transverse center line at a first set position, the transverse center line is perpendicular to the longitudinal center line, and the barbs on two sides of the transverse center line extend towards opposite directions.

The present invention provides a medical suture device at the same time, which comprises the medical suture according to one of the embodiments above, and further comprises a suture needle arranged at a first end of the medical suture and a tail ring arranged at a second end of the medical suture; and the medical suture comprises a barb section located at a middle part and provided with a barb structure, and guide sections located at end parts and not provided with a barb structure, and the suture needle and the medical suture, and the tail ring and the medical suture are in transition through the guide section.

The present invention provides a medical suture device at the same time, which comprises the medical suture according to one of the embodiments above, and suture needles respectively arranged at two ends of the medical suture; and the medical suture comprises a first barb section and a second barb section with a barb structure, wherein the first barb section and the second barb section are connected through a transition section, and guide sections located at end parts and not provided with a barb structure, wherein the suture needles and the medical suture are in transition through the guide sections.

Compared with the prior art, the technical solutions of the present invention have the following technical effects:

In the medical suture and the medical suture device provided by the present invention, compared with an existing suture with the same specification, a transverse distance between tail ends of each pair of barbs is small, and a contact area with tissues along the direction of the longitudinal center line of the suture is reduced by nearly 50%, which is more beneficial for the barb threads to penetrate into the tissues, and beneficial to unidirectionally slide in the tissues, and meanwhile, a cut area of the tissues can be reduced, thus reducing tissue trauma.

### BRIEF DESCRIPTION OF THE DRAWINGS

The preferred embodiments in the present invention will be described in detail hereinafter with reference to the drawings, which will be helpful to understand the objects and advantages of the present invention, wherein:

FIG. 1 is a stereoscopic diagram of a specific embodiment of a medical suture provided by the present invention;

FIG. 2 is a schematic structural diagram of the specific embodiment of the medical suture provided by the present invention;

FIG. 3 is a schematic structural diagram of a medical suture device of a unidirectional equidistant medical suture provided by the present invention;

FIG. 4 is a schematic structural diagram of a medical suture device of a bidirectional equidistant medical suture provided by the present invention;

FIG. 5 is a schematic structural diagram of a medical suture device of a unidirectional unequidistant medical suture provided by the present invention; and

FIG. 6 is a schematic structural diagram of a medical suture device of a bidirectional unequidistant medical suture provided by the present invention.

### DETAILED DESCRIPTION

The technical solutions of the present invention are clearly and completely described hereinafter with reference to the drawings. Obviously, the described embodiments are only some but not all of the embodiments of the present invention. Based on the embodiments in the present invention, all other embodiments obtained by those of ordinary skills in the art without going through any creative work should fall within the scope of protection of the present invention.

In the description of the present invention, it should be noted that the orientation or position relationship indicated by the terms “center”, “up”, “down”, “left”, “right”, “vertical”, “horizontal”, “inside”, “outside”, and the like is based on the orientation or position relationship shown in the drawings, it is only for the convenience of description of the present invention and simplification of the description, and it is not to indicate or imply that the indicated device or element must have a specific orientation, and be constructed and operated in a specific orientation. Therefore, the terms should not be understood as limiting the present invention. Moreover, the terms “first”, “second” and “third” are used for descriptive purposes only and cannot be understood as indicating or implying relative importance.

In the description of the present invention, it should be noted that the terms “installation”, “connected” and “connection” should be understood in a broad sense unless otherwise specified and defined explicitly. For example, they may be fixed connection, removable connection or integrated connection; and may be direct connection, or indirect connection through an intermediate medium, and connection inside two elements. The specific meanings of the above terms in the present invention can be understood in a specific case by those of ordinary skills in the art.

In addition, the technical features involved in different embodiments of the present invention described hereinafter may be combined with each other as long as they do not conflict with each other.

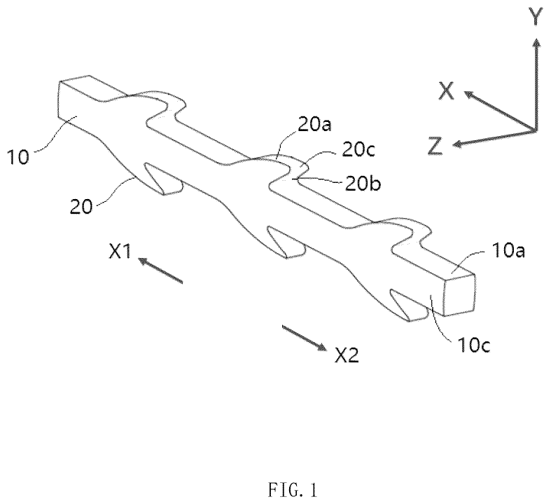

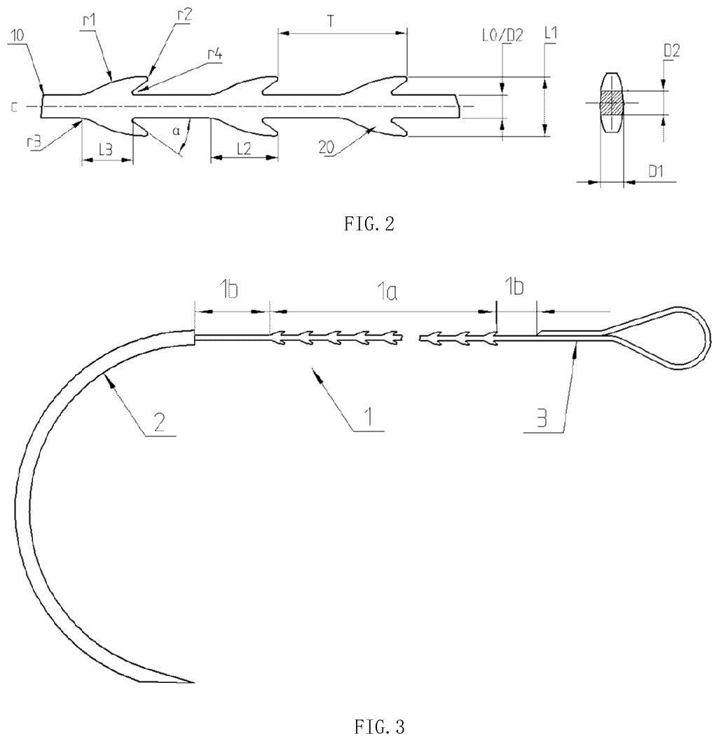

FIG. 1 and FIG. 2 show a specific embodiment of a medical suture provided by the present invention, and the medical suture is used for seaming wounds, closing tissues, tensioning tissues and attaching a repair device. The suture comprises a suture body 10 and a plurality of pairs of barbs 20 integrally formed on the suture body 10, wherein the barb 20 is designed to be convenient to penetrate through tissues when pulled along a first direction X1, and to fit the tissues when pulled along an opposite second direction X2, thus tensioning the tissues.

The suture body 10 defines a longitudinal center line c, and the barbs 20 are arranged at intervals along the longitudinal center line c. Each pair of barbs 20 is symmetrically arranged on opposite sides of the suture body 10, the barb 20 is provided with a first end (which may also be called a connecting end) connected with the suture body 10 and a second end (which may also be called a tail end or a free end) far away from the suture body 10, and a ratio L1/L0 of a transverse distance L1 of the second ends of each pair of the barbs 20 along a direction perpendicular to the longitudinal center line c to a transverse dimension L0 of the suture body 10 perpendicular to the direction of the longitudinal center line c ranges from 1.5 to 2.5.

Compared with the existing suture with the same specification (based on a cross-sectional dimension of the suture body 10), a transverse distance between tail ends of each pair of barbs of the suture above is small, and a contact area of the barb with tissues along an X direction (which is namely the direction of the longitudinal center line c) is reduced by nearly 50%, which is more beneficial for the barb thread to penetrate into the tissues, and beneficial to unidirectionally slide in the tissues, and meanwhile, a cut area of the tissues can be reduced, thus reducing tissue trauma.

In an optional embodiment, a cross section of the suture body 10 in an area without the barbs 20 is approximately rectangular, and the suture body 10 comprises a first end face 10a and a second end face which are oppositely arranged and a third end face 10c and a fourth end face which are oppositely arranged, wherein a plane formed by an X direction and a Z direction is parallel to the first end face 10a and the second end face, and a plane formed by the X direction and a Y direction is parallel to the third end face 10c and the fourth end face. The barbs 20 are connected onto the first end face 10a and the second end face, a width D1 of the first end face 10a ranges from 0.2 mm to 0.6 mm, and a width D2 (which is namely a transverse dimension L0 of the suture body 10 perpendicular to the direction of the longitudinal center line c) of the third end face 10c ranges from 0.2 mm to 0.6 mm.

Preferably, the first end face 10a and the third end face 10c have an equal width, so that a cross section of the suture body 10 in an area without the barbs 20 is approximately square, thus making the suture body of the suture have a good bending moment consistency around Y and Z axes.

A cross section of the suture body 10 in an area with the barbs 20 is approximately spindle-shaped, which means that a cross section of the barb is gradually reduced from the suture body 10 to the tail end, which is beneficial for the barb to penetrate into tissues, and further reducing a cut area of the tissues, thus reducing tissue trauma.

Specifically, the barb 20 comprises an outside face 20a located on an outer side and an inside face 20b located on an inner side. During tissue suturing with the suture, the outside face 20a penetrates through a first side of the tissues to a second side of the tissues along with the suture body 10, while the inside face 20b abuts on the second side of the tissues after the whole barb 20 penetrates through the tissues. The outside face 20a extends to the second end of the barb 20 along a first part of the suture body 10, the inside face 20b extends to the second end of the barb 20 along a second part of the suture body 10, and a ratio of a longitudinal dimension L2 of the barb 20 along the direction of the longitudinal center line c to the transverse dimension L0 of the suture body 10 perpendicular to the direction of the longitudinal center line c ranges from 1.8 to 2.8.

When the suture body is pulled in hard tissues (such as fascia and joint capsule) in a direction reversing X, an existing common barb structure is prone to elastic deformation due to a long arm of force, thus affecting a fixing effect of the tissues. However, the barb 20 in the embodiment has small transverse and longitudinal dimensions, a high structural strength, and a better grasping effect on the tissues. Meanwhile, since the barb 20 has the small transverse and longitudinal dimensions, a bending moment of the suture body of the suture around Y and Z axes is small, so that a bending radius of the suture body around the Y and Z axes is smaller, and the suture is easier to bend compared with an existing suture. Therefore, the suture body has a good compliance in both Y and Z directions, and the suture may better fit the tissues in vivo.

More specifically, a longitudinal dimension L2 of the barb 20 along the direction of the longitudinal center line c, which is namely a distance from the first part of the suture body 10 to the second end of the barb 20 along the direction of the longitudinal center line c, ranges from 0.6 mm to 1.2 mm.

A distance L3 between the first part and the second part along the direction of the longitudinal center line c ranges from 0.4 mm to 1 mm. Compared with the suture body of the same specification, each barb 20 of the suture has a higher base structure strength, and under the same stress condition, the barb 20 is less likely to deform than the barb 20 of the existing suture.

In an optional embodiment, the outside face 20a is approximately an arc face, and the inside face 20b is approximately a straight face. By setting the outside face 20a of the barb 20 as the arc face, it is convenient for the suture to penetrate into the tissues, so that the barb 20 unidirectionally slides in the tissues more smoothly; and by setting the inside face 20b of the barb 20 as the straight face structure, the barb 20 fits the tissues more closely, so that a grasping force to the tissues is greater and more stable. More specifically, a radius r1 of the arc face of the outside face ranges from 1 mm to 2 mm. An included angle α between an extension face of the inside face 20b and the longitudinal center line c ranges from 25 degrees to 45 degrees.

In an optional embodiment, the outside face 20a and the inside face 20b are in transition through a first arc face 20c, and a radius r2 of the first arc face 20c ranges from 0.02 mm to 0.05 mm. Compared with the structure of the barb 20 of the existing suture, the first arc face 20c at the tail end of the barb 20 in the embodiment is small, and the barb is easier to penetrate into the tissues through a design of similar sharp angle, thus being beneficial for the suture body to fix the tissues in vivo.

In an optional embodiment, the outside face 20a and the suture body 10 are in transition through a second arc face, and a radius r3 of the second arc face ranges from 0.5 mm to 1.5 mm. The inside face 20b and the suture body 10 are in transition through a third arc face, and a radius r4 of the third arc face ranges from 0.02 mm to 0.05 mm. Therefore, the barb 20 is matched with the tissues more smoothly after initially penetrating into the tissues and penetrating out of the tissues, thus reducing tissue trauma.

With reference to FIG. 3 and FIG. 5, the present invention provides a specific embodiment of a medical suture device, and the medical suture device comprises a medical suture 1, a suture needle 2 arranged at a first end of the medical suture 1, and a tail ring 3 arranged at a second end of the medical suture 1. Each of the barbs 20 of the medical suture 1 arranged at intervals along the longitudinal center line c extends in the same direction to form a unidirectional barb suture 1. The medical suture 1 comprises a barb section 1a located at a middle part and provided with a barb structure, and guide sections 1b located at end parts and not provided with a barb structure, and the suture needle 2 and the medical suture 1, and the tail ring 3 and the medical suture 1 are in transition through the guide section 1b.

In an optional embodiment, with reference to FIG. 3, the barbs 20 are equidistantly distributed along an extension direction of the longitudinal center line c, and a distance T between adjacent barbs 20 ranges from 1 mm to 2.5 mm. In another optional embodiment, with reference to FIG. 5, according to a specific tissue to be sutured, the barbs 20 are unequidistantly distributed along the extension direction of the longitudinal center line c, and the distance T between the adjacent barbs 20 ranges from 1 mm to 2.5 mm, so that the barbs 20 have a better adaptability with the specific tissue to be sutured.

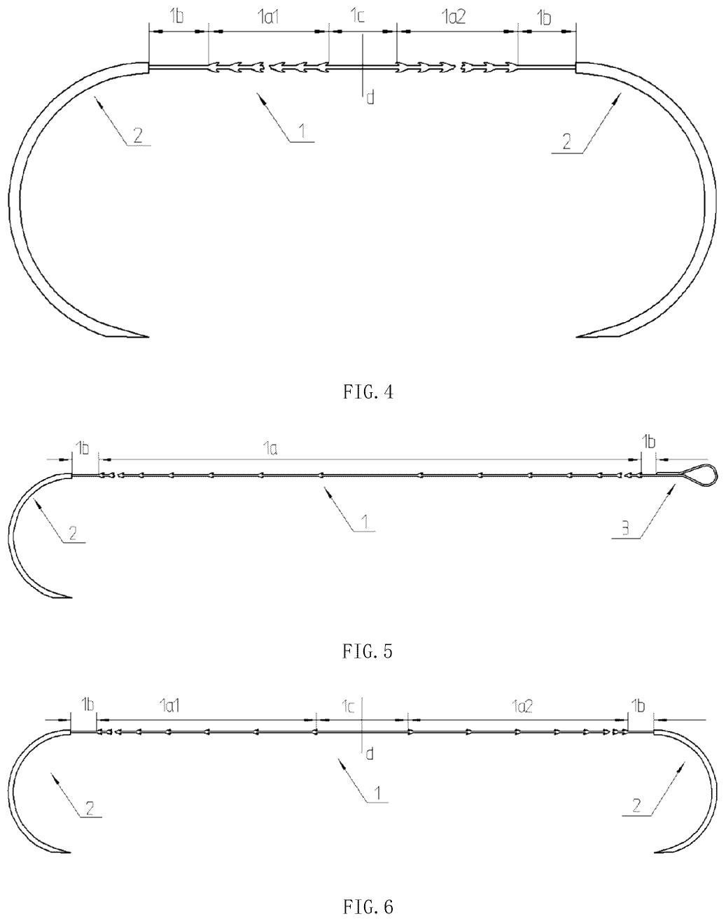

With reference to FIG. 4 and FIG. 6, the present invention provides another specific embodiment of a medical suture device, and the medical suture device comprises a medical suture 1 and suture needles 2 respectively arranged at two ends of the medical suture 1. The suture body 10 of the medical suture 1 forms a transverse center line at a first set position d, the transverse center line d is perpendicular to the longitudinal center line c, and the barbs 20 located on two sides of the transverse center line d extend in opposite directions to form a bidirectional barb suture 1. The medical suture 1 comprises a first barb section 1a1 and a second barb section 1a2 with a barb structure, wherein the first barb section 1a1 and the second barb section 1a2 are connected through a transition section 1c, and guide sections 1b located at end parts and not provided with a barb structure, wherein the barbs of the first barb section 1a1 and the second barb section 1a2 are oppositely arranged, and the suture needles 2 and the medical suture 1 are in transition through the guide sections 1b.

In an optional embodiment, with reference to FIG. 4, the barbs 20 are equidistantly distributed along an extension direction of the longitudinal center line c, and a distance T between adjacent barbs 20 ranges from 1 mm to 2.5 mm. In another optional embodiment, with reference to FIG. 6, according to a specific tissue to be sutured, the barbs 20 are unequidistantly distributed along the extension direction of the longitudinal center line c, and the distance T between the adjacent barbs 20 ranges from 1 mm to 2.5 mm, so that the barbs 20 have a better adaptability with the specific tissue to be sutured.

Obviously, the above embodiments are only examples for the purpose of clear illustration, but are not intended to limit the embodiments. For those of ordinary skills in the art, other different forms of changes or variations may be made on the basis of the above illustration. It is not necessary or possible to exhaust all the embodiments here. Moreover, the obvious changes or variations derived from this are still included within the scope of protection of the present invention.

1. A medical suture, comprising:
a suture body, wherein the suture body defines a longitudinal center line; and
a plurality of pairs of barbs arranged at intervals along a direction of the longitudinal center line, wherein each pair of barbs is symmetrically arranged on opposite sides of the suture body, the barb is provided with a first end connected with the suture body and a second end far away from the suture body, and a ratio of a transverse distance L1 of second ends of each pair of the barbs along a direction perpendicular to the longitudinal center line to a transverse dimension L0 of the suture body perpendicular to the direction of the longitudinal center line ranges from 1.5 to 2.5.

2. The medical suture according to claim 1, wherein a cross section of the suture body in an area without the barbs is approximately rectangular, and the suture body comprises a first end face and a second end face which are oppositely arranged and a third end face and a fourth end face which are oppositely arranged, wherein the barbs are connected onto the first end face and the second end face, a width D1 of the first end face ranges from 0.2 mm to 0.6 mm, and a width D2 of the third end face ranges from 0.2 mm to 0.6 mm.

3. The medical suture according to claim 2, wherein the cross section of the suture body in the area without the barbs is approximately square.

4. The medical suture according to claim 2, wherein a cross section of the suture body in an area with the barbs is approximately spindle-shaped.

5. The medical suture according to claim 1, wherein the barb comprises an outside face and an inside face, the outside face extends to the second end of the barb along a first part of the suture body, the inside face extends to the second end of the barb along a second part of the suture body, and a ratio of a longitudinal dimension L2 of the barb along the direction of the longitudinal center line to the transverse dimension L0 of the suture body perpendicular to the direction of the longitudinal center line ranges from 1.8 to 2.8.

6. The medical suture according to claim 5, wherein a distance L3 between the first part and the second part along the direction of the longitudinal center line ranges from 0.4 mm to 1 mm.

7. The medical suture according to claim 5, wherein the outside face is approximately an arc face, and the inside face is approximately a straight face.

8. The medical suture according to claim 7, wherein a radius r1 of the arc face of the outside face ranges from 1 mm to 2 mm.

9. The medical suture according to claim 7, wherein an included angle between an extension face of the inside face and the longitudinal center line ranges from 25 degrees to 45 degrees.

10. The medical suture according to claim 5, wherein the outside face and the inside face are in transition through a first arc face, and a radius r2 of the first arc face ranges from 0.02 mm to 0.05 mm.

11. The medical suture according to claim 5, wherein the outside face and the suture body are in transition through a second arc face, and a radius r3 of the second arc face ranges from 0.5 mm to 1.5 mm.

12. The medical suture according to claim 5, wherein the inside face and the suture body are in transition through a third arc face, and a radius r4 of the third arc face ranges from 0.02 mm to 0.05 mm.

13. The medical suture according to claim 1, wherein the barbs are equidistantly distributed along an extension direction of the longitudinal center line, and a distance T between adjacent barbs ranges from 1 mm to 2.5 mm.

14. The medical suture according to claim 1, wherein the barbs are unequidistantly distributed along an extension direction of the longitudinal center line, and a distance T between adjacent barbs ranges from 1 mm to 2.5 mm.

15. The medical suture according to claim 1, wherein the barbs extend towards the same direction to form a unidirectional barb suture.

16. The medical suture according to claim 1, wherein the suture body forms a transverse center line at a first set position, the transverse center line is perpendicular to the longitudinal center line, and the barbs on two sides of the transverse center line extend towards opposite directions.

17. A medical suture device, comprising the medical suture according to claim 1, and further comprising a suture needle arranged at a first end of the medical suture and a tail ring arranged at a second end of the medical suture;
the medical suture comprising a barb section located at a middle part and provided with a barb structure, and guide sections located at end parts and not provided with a barb structure, and the suture needle and the medical suture, and the tail ring and the medical suture being in transition through the guide section.

18. A medical suture device, comprising the medical suture according to claim 16, and suture needles respectively arranged at two ends of the medical suture;
the medical suture comprising a first barb section and a second barb section with a barb structure, the first barb section and the second barb section being connected through a transition section; and guide sections located at end parts and not provided with a barb structure, the suture needles and the medical suture being in transition through the guide sections.

19. The medical suture device according to claim 17, wherein a cross section of the suture body in an area without the barbs is approximately rectangular, and the suture body comprises a first end face and a second end face which are oppositely arranged and a third end face and a fourth end face which are oppositely arranged, wherein the barbs are connected onto the first end face and the second end face, a width D1 of the first end face ranges from 0.2 mm to 0.6 mm, and a width D2 of the third end face ranges from 0.2 mm to 0.6 mm.

20. The medical suture device according to claim 17, wherein the barb comprises an outside face and an inside face, the outside face extends to the second end of the barb along a first part of the suture body, the inside face extends to the second end of the barb along a second part of the suture body, and a ratio of a longitudinal dimension L2 of the barb along the direction of the longitudinal center line to the transverse dimension L0 of the suture body perpendicular to the direction of the longitudinal center line ranges from 1.8 to 2.8.
